(19) United States
(12) Patent Application Publication
(10) Pub. No.: US 2019/0321651 A1
(43) Pub. Date: Oct. 24, 2019

(54) System for Optical Stimulation of Target Cells

(71) Applicant: The Board of Trustees of the Leland Stanford Junior University, Stanford, CA (US)

(72) Inventors: Karl Deisseroth, Stanford, CA (US); Michael Bret Schneider, Portola Valley, CA (US); David J. Mishelevich, Playa Del Rey, CA (US)

(21) Appl. No.: 16/404,430

(22) Filed: May 6, 2019

Related U.S. Application Data
(63) Continuation of application No. 14/537,305, filed on Nov. 10, 2014, now abandoned.
Application No. 14/537,305 is a continuation of application No. 11/651,422, filed on Jan. 9, 2007, now Pat. No. 8,926,959.
Application No. 11/651,422 is a continuation-in-part of application No. 11/459,636, filed on Jul. 24, 2006, now Pat. No. 8,906,360.
(60) Provisional application No. 60/701,799, filed on Jul. 22, 2005.

(51) Int. Cl. A61N 5/06; A61K 48/00; A61K 41/00; A61K 38/17

(52) U.S. Cl. CPC A61N 5/062; A61K 48/0058; A61K 41/0057; A61K 38/177; A61K 48/0075; A61N 2005/0651; A61N 5/0622; A61N 5/0601; A61K 48/0083; Y10S 530/825; A61K 48/005

(57) ABSTRACT

Stimulation of target cells using light, e.g., in vivo, is implemented using a variety of methods and devices. According to an example embodiment of the present invention, target cells are stimulated using an implantable arrangement. The arrangement includes an electrical light-generation means for generating light and a biological portion. The biological portion has a photosensitive bio-molecular arrangement that responds to the generated light by stimulating target cells in vivo.

RELATED PATENT DOCUMENTS

This patent document is a continuation-in-part of U.S. patent application Ser. No. 11/459,636 (now U.S. Pat. No. ______) filed on Jul. 24, 2006 and entitled “Light-Activated Cation Channel and Uses Thereof,” which is fully incorporated herein by reference and to which priority is claimed under 35 U.S.C. § 120 for common subject matter; this patent document claims further benefit of U.S. Provisional Application No. 60/701,799 filed Jul. 22, 2005.

### FIELD OF THE INVENTION

The present invention relates generally to systems and approaches for stimulating target cells, and more particularly to using an optical device to stimulate the target cells.

### BACKGROUND

The stimulation of various cells of the body has been used to produce a number of beneficial effects. One method of stimulation involves the use of electrodes to introduce an externally generated signal into cells. One problem faced by electrode-based brain stimulation techniques is the distributed nature of neurons responsible for a given mental process. Conversely, different types of neurons reside close to one another such that only certain cells in a given region of the brain are activated while performing a specific task. Alternatively stated, not only do heterogeneous nerve tracts move in parallel through tight spatial confines, but the cell bodies themselves may exist in mixed, sparsely embedded configurations. This distributed manner of processing seems to defy the best attempts to understand canonical order within the CNS, and makes neuromodulation a difficult therapeutic endeavor. This architecture of the brain is poses a problem for electrode-based stimulation because electrodes are relatively indiscriminate with regards to the underlying physiology of the neurons that they stimulate. Instead, physical proximity of the electrode poles to the neuron is often the single largest determining factor as to which neurons will be stimulated. Accordingly, it is generally not feasible to absolutely restrict stimulation to a single class of neuron using electrodes.

Another issue with the use of electrodes for stimulation is that because electrode placement dictates which neurons will be stimulated, mechanical stability is frequently inadequate, and results in lead migration of the electrodes from the targeted area. Moreover, after a period of time within the body, electrode leads frequently become encapsulated with glial cells, raising the effective electrical resistance of the electrodes, and hence the electrical power delivery required to reach targeted cells. Compensatory increases in voltage, frequency or pulse width, however, may spread of electrical current may increase the unintended stimulation of additional cells.

Another method of stimulus uses photosensitive bio-molecular structures to stimulate target cells in response to light. For instance, light activated proteins can be used to control the flow of ions through cell membranes. By facilitating or inhibiting the flow of positive or negative ions through cell membranes, the cell can be briefly depolarized, depolarized and maintained in that state, or hyperpolarized. Neurons are an example of a type of cell that uses the electrical currents created by depolarization to generate communication signals (i.e., nerve impulses). Other electrically excitable cells include skeletal muscle, cardiac muscle, and endocrine cells. Recently discovered techniques allow for stimulation of cells resulting in the rapid depolarization of cells (e.g., in the millisecond range). Such techniques can be used to control the depolarization of cells such as neurons. Neurons use rapid depolarization to transmit signals throughout the body and for various purposes, such as motor control (e.g., muscle contractions), sensory responses (e.g., touch, hearing, and other senses) and computational functions (e.g., brain functions). Thus, the control of the depolarization of cells can be beneficial for a number of different purposes, including (but not limited to) psychological therapy, muscle control and sensory functions. For further details on specific implementations of photosensitive bio-molecular structures and methods, reference can be made to “Millisecond-Timescale, Genetically Optical Control of Neural Activity”, by Boyden, Edward S. et al., Nature Neuroscience 8, 1263-1268 (2005). This reference discusses use of blue-light-activated ion channel channelrhodopsin-2 (ChR2) to cause calcium (Ca++)-mediated neural depolarization, and is fully incorporated herein be reference. Other applicable light-activated ion channels include halorhodopsin (NpHR), in which amber light affects chloride (Cl−) ion flow so as to hyperpolarize neuronal membrane, and make it resistant to firing.

While these and other methods are promising scientific discoveries, there is need for innovations that allow for practical application of these basic mechanisms, such as in vivo neuromodulation, for example, to treat diseases in humans. Often, the specific location at which the photosensitive bio-molecular structure is applied to is critical. Moreover, the process by which light is made able to reach the photosensitive bio-molecular structures can involves obstacles, on the practical level. In many applications, minimal invasiveness of the procedure is paramount. For instance, the brain is a delicate organ and less disruption is usually a paramount issue for surgeries and similar procedures on the brain. Thus, it is sometimes desirable that the extent of any surgical procedure be kept to a minimum. This can be difficult, however, where large devices are needed for the administration of treatment. In some applications the comfort of the patient is also important. Thus, external apparatus can be less than ideal.

These and other issues have presented challenges to the implementation of the stimulus of target cells, including those involving photosensitive bio-molecular structures and those used in similar applications.

### SUMMARY

The claimed invention is directed to photosensitive bio-molecular structures and related methods. The present invention is exemplified in a number of implementations and applications, some of which are summarized below.

According to one example embodiment of the present invention, an implantable arrangement is implemented having a light-generation device for generating light. The arrangement also has a biological portion that modifies target cells for stimulation in response to light generated by the light-generation means in vivo.

According to another example embodiment of the present invention, target cells are stimulated using an implantable arrangement. The arrangement includes an electrical light-generation means for generating light and a biological portion. The biological portion has a photosensitive bio-molecular arrangement that responds to the generated light by stimulating target cells in vivo. Stimulation may be manifest as either up-regulation, or down-regulation of activity at the target.

According to another example embodiment of the present invention, an implantable device delivers gene transfer vector, such as a virus, which induces expression of photosensitive bio-molecular membrane proteins. The device has a light generator, responsive to (for example, charged by or triggered by) an external signal, to generate light and a biological arrangement that includes the photosensitive bio-molecular protein that responds to the generated light by interacting with target cells in vivo. In this manner, the electronic portions of the device may be used to optically stimulate target cells. Stimulation may be manifest as either upregulation (e.g. increased neuronal firing activity), or downregulation (e.g. neuronal hyperpolarization, or alternatively, chronic depolarization) of activity at the target.

According to another example embodiment of the present invention, a method is implemented for stimulating target cells using photosensitive proteins that bind with the target cells. The method includes a step of implanting the photosensitive proteins and a light generating device near the target cells. The light generating device is activated and the photosensitive protein stimulates the target cells in response to the generated light.

Applications include those associated with any population of electrically-excitable cells, including neurons, skeletal, cardiac, and smooth muscle cells, and insulin-secreting pancreatic beta cells. Major diseases with altered excitation-effector coupling include heart failure, muscular dystrophies, diabetes, pain, cerebral palsy, paralysis, depression, and schizophrenia. Accordingly, the present invention has utility in the treatment of a wide spectrum of medical conditions, from Parkinson's disease and brain injuries to cardiac dysrhthmias, to diabetes, and muscle spasm.

The above summary of the present invention is not intended to describe each illustrated embodiment or every implementation of the present invention. The figures and detailed description that follow more particularly exemplify these embodiments.

### BRIEF DESCRIPTION OF THE DRAWINGS

The invention may be more completely understood in consideration of the detailed description of various embodiments of the invention that follows in connection with the accompanying drawings, in which:

FIG. 1 shows a block diagram of a system for stimulating target cells, according to an example embodiment of the present invention;

FIG. 2 shows a block diagram of an implantable device for stimulating target cells, according to an example embodiment of the present invention;

FIG. 3 shows a block diagram of an implantable device, according to an example embodiment of the present invention;

FIG. 4A shows a block diagram of an implantable device, according to an example embodiment of the present invention;

FIG. 4B shows a circuit diagram corresponding to the block diagram of FIG. 4A, according to an example embodiment of the present invention;

FIG. 5A and FIG. 5B show a diagram of a mesh for containing photosensitive bio-molecules, according to an example embodiment of the present invention;

FIG. 6A and FIG. 6B show a diagram of a viral matrix, according to an example embodiment of the present invention;

FIG. 7 shows a circuit diagram of a circuit that produces light in response to a magnetic field, according to an example embodiment of the present invention;

FIG. 8A-8C show a block diagram and circuits for the production of light in response to a RF signal, according to an example embodiment of the present invention;

FIG. 9A and FIG. 9B each show a diagram of a fiber-optic device, according to an example embodiment of the present invention;

FIGS. 10A-10D depict various stages in the production of a photosensitive biological portion, according to an example embodiment of the present invention;

FIG. 11 shows an implantation device, according to an example embodiment of the present invention; and

FIG. 12A and FIG. 12B show a diagram for another implantation device, according to an example embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The present invention is believed to be useful for enabling practical application of a variety of photosensitive bio-molecular structures, and the invention has been found to be particularly suited for use in arrangements and methods dealing with neuron stimulation. While the present invention is not necessarily limited to such applications, various aspects of the invention may be appreciated through a discussion of various examples using this context.

Consistent with one example embodiment of the present invention, target cells are stimulated using an implantable arrangement. The implantable arrangement includes a biological portion that facilitates the stimulation of the target cells in response to receipt of light. The implantable arrangement also includes a light generator for creating light to trigger the stimulus of the target cells.

Consistent with another example embodiment of the present invention, a method is implemented for stimulating target cells in vivo using gene transfer vectors (for example, viruses) capable of inducing photosensitive ion channel growth (for example, ChR2 ion channels). The vectors are implanted in the body, along with the electronic components of the apparatus. A light producing device is implanted near the target cells. The target cells are stimulated in response to light generated by the light producing device.

As used herein, stimulation of a target cell is generally used to describe modification of properties of the cell. For instance, the stimulus of a target cell may result in a change in the properties of the cell membrane that can lead to the depolarization or polarization of the target cell. In a particular instance, the target cell is a neuron and the stimulus affects the transmission of impulses by facilitating or inhibiting the generation of impulses by the neuron.

Consistent with another example embodiment of the present invention, the target cells are neurons located in the brain of a mammal. The target cells are genetically modified to express photosensitive bio-molecular arrangement, for example, ChR2 ion channels. Light can then be used to stimulate the neurons. Depending upon a number of factors, such as the location within the brain and the frequency and length of stimulation, different objectives can be achieved. For instance, current techniques for deep brain stimulus (DBS) use electrodes to apply a current directly to the targeted area of the brain. The frequency of the electrical stimulus is sometimes referred to as either low-frequency DBS or high-frequency DBS. Studies have suggested that high-frequency DBS inhibits the generation of impulses from the stimulated cells, while low-frequency DBS facilitates the generation of impulses from the stimulated cells. The frequencies that produce the effects of high-frequency of low-frequency DBS have also been shown to vary depending upon the specific area of the brain being stimulated. According to one example of high-frequency DBS, the neurons are stimulated using electrodes supplying current pulses at frequencies around 100 Hz or more. Such a frequency has been shown to be effective in certain applications, as discussed further herein.

A specific example of DBS is used for the treatment of Parkinson's disease. In this application, DBS is often applied to the globus pallidus interna, or the subthalamic nucleus within a patient's brain. By implanting a biological arrangement that modifies the cells to respond to light, a light flashing light can be used in place of electrodes. Thus, the targeted neuron cells and external electrical signal need not be directly applied to the targeted cells. Moreover, light can often travel from its point of origin farther than electricity, thereby increasing the effective area relative to the stimulation source and only those neurons that have been photosensitized are stimulated.

As with the electrode-based DBS methods, one embodiment of the present invention can be implemented using high-frequency DBS to inhibit neuron generated impulses. While high-frequency DBS has been accomplished at frequencies around 100 Hz, high-frequency DBS using various embodiments of the present invention may not necessarily require the same frequency. For instance, it may be possible to reproduce the inhibiting effects of high-frequency DBS at lower frequencies (e.g., 50 Hz) when using light activated techniques. For example, activation of the halorhodopsin (NpHR) channel intrinsically favors hyperpolarization and resistance to action potential generation. Also, a light-sensitive ion channel may recover more slowly than naturally occurring mammalian ion channels, thus slowing the repolarization (and hence overall reactivity) of a neuron. Thus, various frequencies can be used depending upon the particular application (e.g., the targeted portion of the brain and the desired effect), and the stimulation modality being applied.

Consistent with another example embodiment of the present invention, gene transfer vectors inducing the expression of photosensitive bio-molecules are used to target a specific type of cell. For instance, viral-based proteins (e.g., lentiviruses or retroviruses) can created to target specific types of cells, based upon the proteins that they uniquely express. The targeted cells are then infected by the viral-based gene-transfer proteins, and begin to produce a new type of ion channel (for example ChR2), thereby becoming photosensitive. This can be particularly useful for stimulating the targeted cells without stimulating other cells that are in proximity to the targeted cells. For example, neurons of disparate length, diameter, chronaxie, other membrane properties, electrical insulation, neurotransmitter output, and overall function, lie in close proximity to one another, and thus, can be inadvertently stimulated when using electrodes to provide the stimulation of the neurons. For further details on the generation of viral vectors, reference may be made to U.S. patent application Ser. No. 11/459,636 filed on Jul. 24, 2006, which is fully incorporated herein by reference.

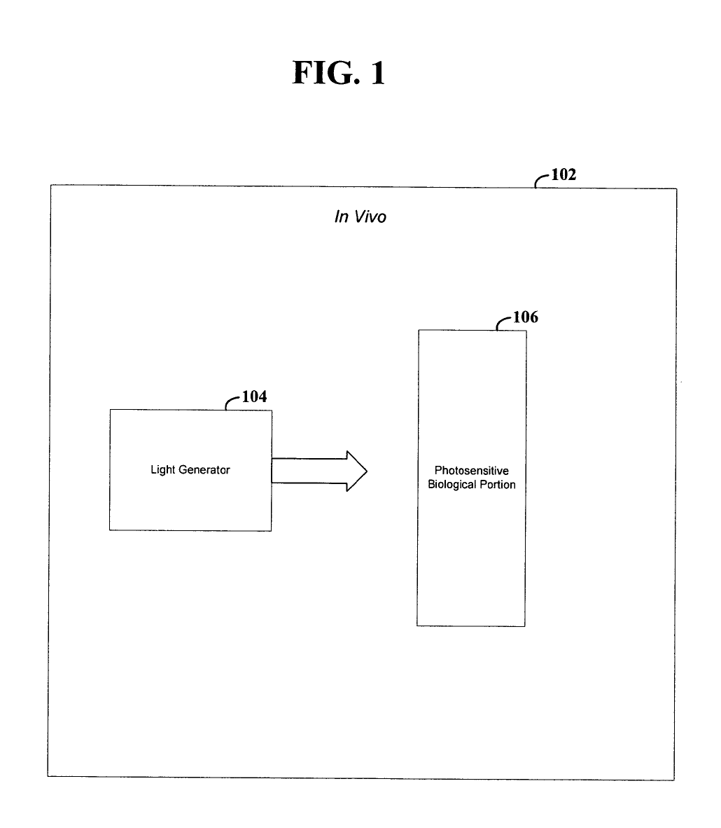

Turning now to the figures, FIG. 1 shows a block diagram of a system for stimulating target cells, according to an example embodiment of the present invention. Block 102 represents a location internal to an organism (e.g., a mammal), as shown by the in vivo designation. Light generator 104 is an implantable device that generates light in vivo. The photosensitive biological portion 106 affects the target cells such that generated light strikes causes stimulation of the target. In one instance, the light generator 104 is a small electronic device on the order of a few millimeters in size. The small size is particularly useful for minimizing the intrusiveness of the device and associated implantation procedure. In another instance, the light generator 104 may include a fiber optic device that can be used to transmit light from an external source to the target cells.

In one embodiment of the present invention, the target cells are modified to contain light-activated ion channel proteins. A specific example of such protein is channelrhodopsin-3 (ChR2), which is a product based upon green alga Chalamydomanas reinhardtii.

These light sensitive proteins can be implanted using a number of different methods. Example methods include, but are not limited to, the use of various delivery devices, such as gelatin capsules, liquid injections and the like. Such methods also include the use of stereotactic surgery techniques such as frames or computerized surgical navigation systems to implant or otherwise access areas of the body.

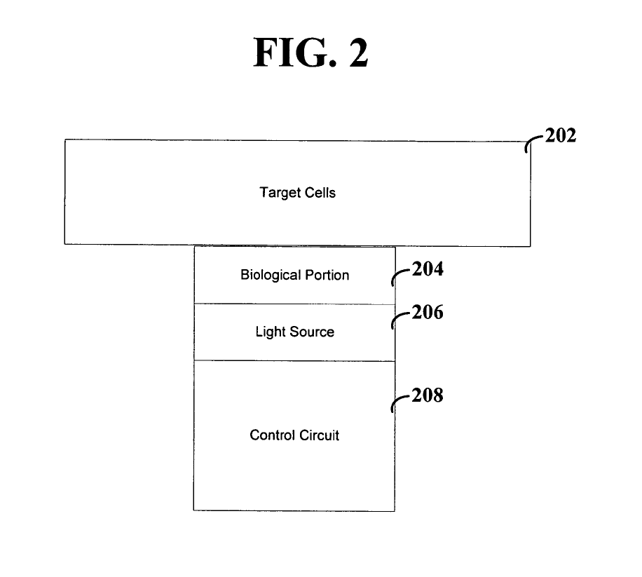

FIG. 2 shows a block diagram of an implantable device for stimulating target cells, according to an example embodiment of the present invention. The figure includes control circuit 208, light source 206, biological portion 204 and target cells 202. Biological portion 204 affects the target cells 202 such that the target cells are stimulated in response to light

In one embodiment of the present invention, biological portion 204 may be composed of target cells 202 that have been modified to be photosensitive. In another embodiment of the present invention, biological portion 204 may contain biological elements such as gene transfer vectors, which cause target cells 202 to become sensitive to light. An example of this is lentiviruses carrying the gene for ChR2 expression. In this manner, the stimulation of target cells 202 can be controlled by the implantable device. For example, the control circuit 208 can be arranged to respond to an external signal by activating, or deactivating light source 206, or by charging the battery that powers light source 206. In one instance, the external signal is electromagnetic radiation that is received by control circuit 208. For example, radio frequency (RF) signals can be transmitted by an external RF transmitter and received by control circuit 208. In another example, a magnetic field can be used to activate and/or power the control circuit.

Control circuit 208 can be implemented using varying degrees of complexity. In one instance, the circuit is a simple coil that when exposed to a magnetic field generates a current. The current is then used to power light source 206. Such an implementation can be particularly useful for limiting the size and complexity as well as increasing the longevity of the device. In another instance, control circuit 208 can include an RF antenna. Optionally, a battery or similar power source, such as a capacitive element, can be used by control circuit 208. While charged, the power source allows the circuitry to continue to operate without need for concurrent energy delivery from outside the body. This can be particularly useful for providing precise control over the light emitted by light source 206 and for increased intensity of the emitted light.

In one embodiment of the present invention, light source 206 is implemented using a light-emitting-diode (LED). LEDs have been proven to be useful for low power applications and also to have a relatively fast response to electrical signals.

In another embodiment of the present invention, biological portion 204 includes a gelatin or similar substance that contains gene transfer vectors which genetically code the target cells for photosensitivity. In one instance, the vectors are released once implanted into the body. This can be accomplished, for example, by using a containment material that allows the vectors to be released into aqueous solution (e.g., using dehydrated or water soluble materials such as gelatins). The release of the vectors results in the target cells being modified such that they are simulated in response to light from light source 206

In another embodiment of the present invention, the biological portion 204 includes a synthetic mesh that contains the photosensitive cells. In one instance, the cells are neurons that have been modified to be photosensitive. The synthetic mesh can be constructed so as to allow the dendrites and axons to pass through the mess without allowing the entire neuron (e.g., the cell body) to pass. One example of such a mesh has pores that are on the order of 3-7 microns in diameter and is made from polyethylene terephthalate. In another example embodiment, the biological portion 204 includes an injection mechanism as discussed in further detail herein.

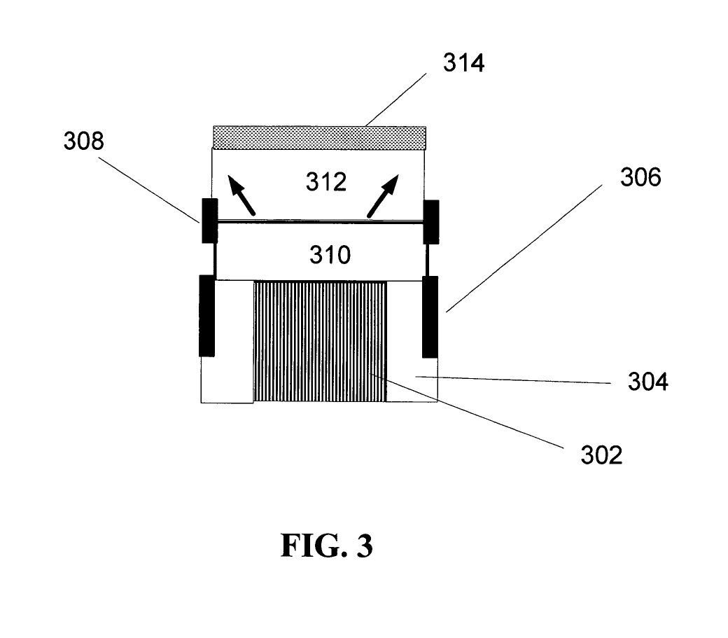

FIG. 3 shows a block diagram of an implantable device, according to an example embodiment of the present invention. The implantable device of FIG. 3 is responsive to a field magnetic. More specifically, an inductor constructed from windings 302 and core 304 generates a current/voltage in response to a magnetic field. The current is passed to control circuit 310 through conductive path 306. In response, control circuit 310 activates light source 312 using conductive path 308. Light source 312 illuminates biological portion 314 in order to stimulate the target cells. In one instance, biological portion 314 includes a gelatin, synthetic mesh or injection mechanism as discussed in further detail herein.

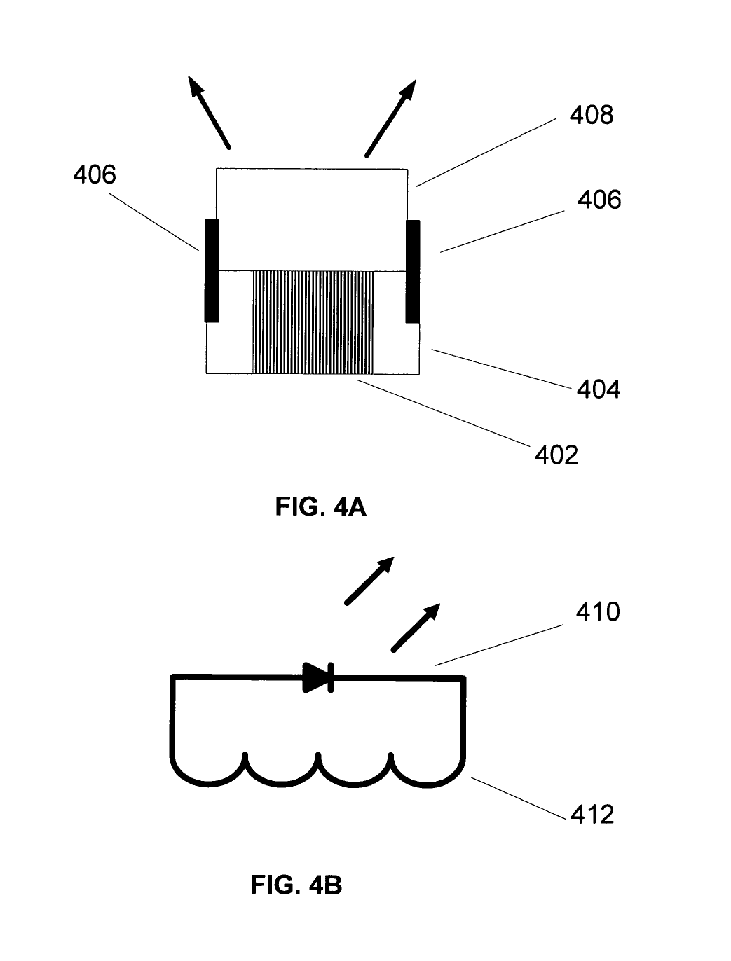

In one embodiment of the present invention, the control portion can be a simple electrical connection, resistive element, or can be removed completely. In such an embodiment, the intensity, duration and frequency of light generated would be directly controlled by the current generated from a magnetic field. This can be particularly useful for creating inexpensive, long lasting and small devices. An example of such an embodiment is discussed further in connection with FIG. 4A and FIG. 4B.

In another embodiment of the present invention, the control portion can be implemented as a more complex circuit. For instance the control circuit may include and otherwise implement different rectifier circuits, batteries, pulse timings, comparator circuits and the like. In a particular example, the control circuit includes an integrated circuit (IC) produced using CMOS or other processes. Integrated circuit technology allows for the use of a large number of circuit elements in a very small area, and thus, a relatively complex control circuit can be implemented for some applications.

In a particular embodiment of the present invention, the inductor (302 and 304) is a surface mount inductor, such as a 100 uH inductor part number CF1008-103K supplied by Gowanda Electronics Corp. The light generating portion is a blue LED, such as LEDs in 0603 or 0805 package sizes. A particular example is a blue surface mount LED having part number SML0805, available from LEDtronics, Inc (Torrance, Calif.). Connective paths 306 and 308 can be implemented using various electrical conductors, such as conductive epoxies, tapes, solder or other adhesive materials. LEDs emitting light in the amber spectrum (as applicable to NpHR channels) are available through commercial sources including this same manufacturer.

FIG. 4A shows a block diagram of an implantable device, according to an example embodiment of the present invention. FIG. 4A shows an inductor comprising coils 402 and core 404 connected to LED 408 using conductive paths shown by 406. FIG. 4B shows a circuit diagram corresponding to the block diagram of FIG. 4A. Inductor 412 is connected in parallel to LED 410. Thus, current and voltage generated by changing a magnetic field seen at inductor 412 causes LED 410 to produce light. The frequency and strength of the changing magnetic field can be varied to produce the desired amount and periodicity of light from LED 410.

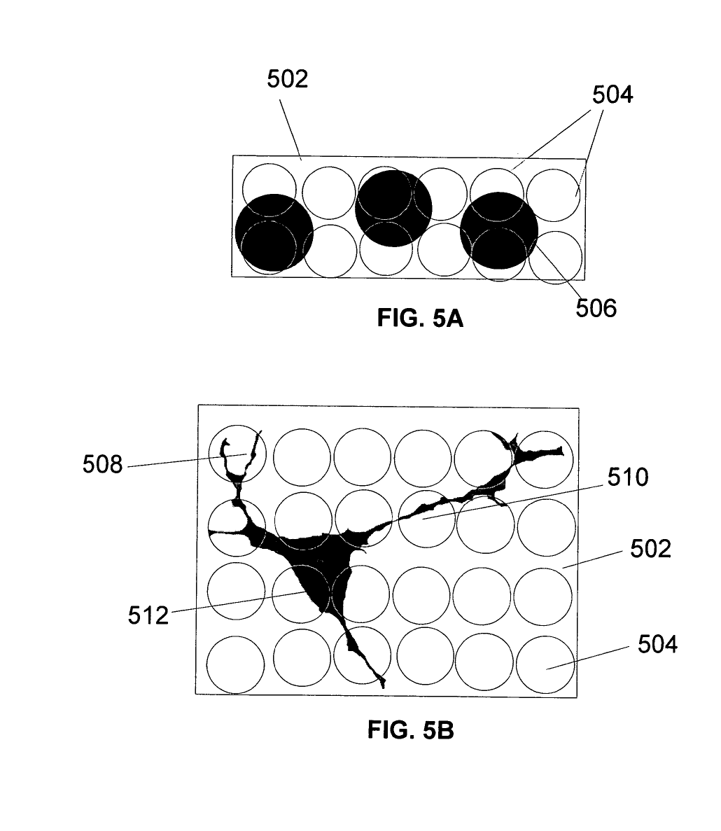

FIG. 5A and FIG. 5B show a diagram of a mesh for containing photosensitive bio-molecules, according to an example embodiment of the present invention. Mesh 502 is constructed having holes 504 of a size that allows illumination to pass but is small enough to prevent cells 506 to pass. This allows for cells 506 to be implanted while still receiving light from a light generator.

In one embodiment of the present invention, the cells 506 are stem cells that are modified to be photosensitive. The stem cells are allowed to mature as shown by FIG. 5B. In a particular instance, the stem cells mature into neurons having a cell body 512, axons/dendrites 508 and 510. The neurons are genetically modified to be photosensitive. Holes 504 are on the order of 3-7 microns in diameter. This size allows some axons and dendrites to pass through holes 504, while preventing the cell body 512 to pass.

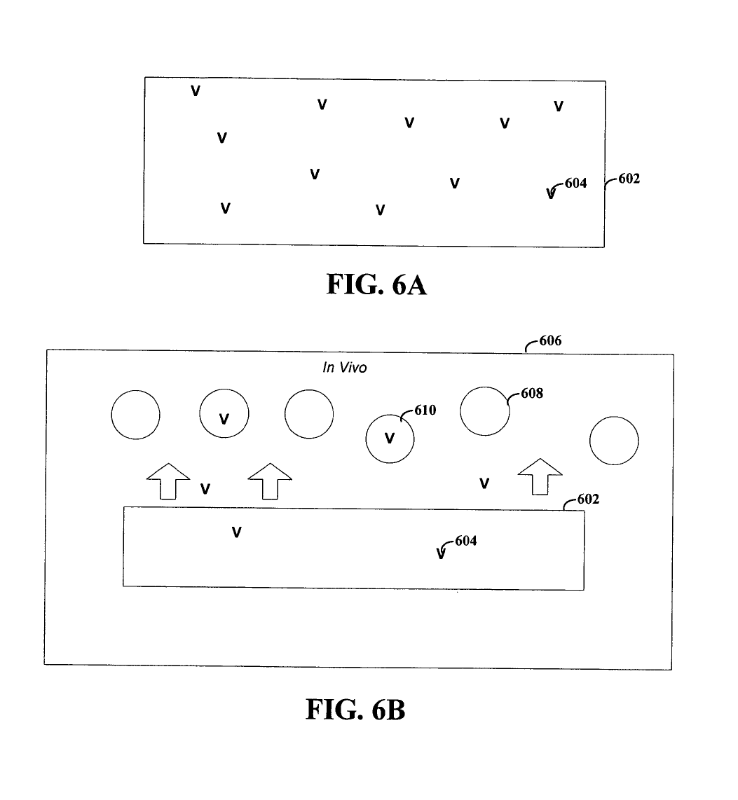

FIG. 6A and FIG. 6B show a diagram of a viral matrix, according to an example embodiment of the present invention. The viral matrix includes structure 602, which contains viral vectors 604. In one instance, structure 602 includes a gel or fluid substance that contains viral vectors 604 until they are implanted in a mammal 606. Once viral vectors 604 are released, they infect target cells 608 in the vicinity of the implanted viral matrix as shown by FIG. 6B. Infected target cell 610 becomes photosensitive, and thus, light can be used to control the stimulation of target cell 610.

According to one embodiment of the present invention, structure 602 is a gelatin that has been impregnated, or otherwise sealed with viral vectors 604 contained within the gelatin. When structure 602 is implanted, the gelatin is hydrated and or dissolved, thereby releasing viral vectors 604. Standard commercially available gelatin mix may be used, in addition to compounds such as Matrigel by BD Biosciences division of Becton Dickenson and Company (Franklin Lakes, N.J.)

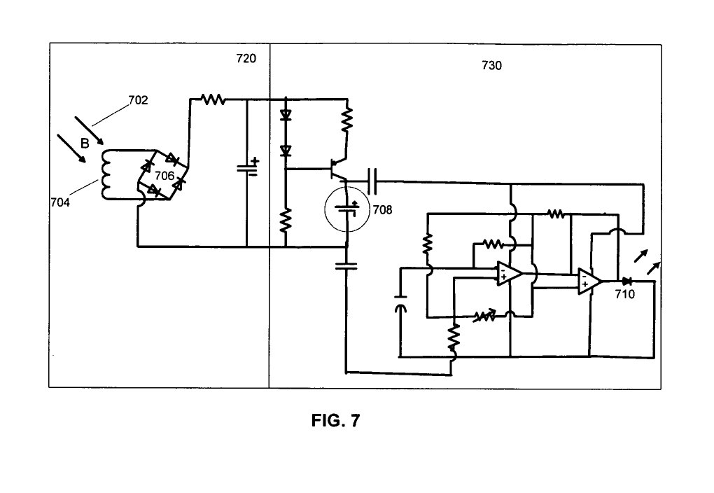

FIG. 7 shows a circuit diagram of a circuit that produces light in response to a magnetic field, according to an example embodiment of the present invention. FIG. 7 includes an input circuit 720 and an output circuit 730. Inductor 704 generates current in response to magnetic field 702. Due to properties of magnetic fields, the current produced by inductor 704 is an alternating current (AC) signal. Full-wave bridge rectifier 706 rectifies the AC signal and along with an RC circuit generates a relatively stable voltage from the AC signal. This generated voltage is responsive to magnetic field 702 and output circuit 730 generates light when the generated voltage is at a sufficient level. More specifically, power from battery 708 is used to drive LED 710 in response to magnetic field 702. This is particularly useful for applications where the magnetic field 702 seen by inductor 704 is less powerful (e.g., due to the in vivo location of inductor 704).

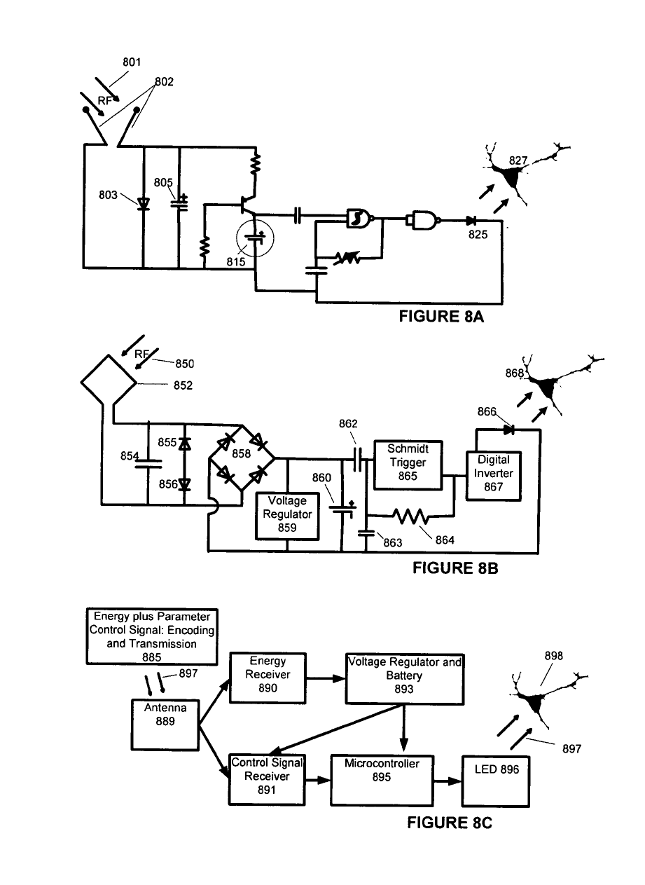

FIG. 8A shows a circuit diagram of a circuit that produces light in response to RF signal 801, according to an example embodiment of the present invention. Antenna 802 is used to receive RF transmission 801 and convert the signal to electricity. The received transmission is rectified by diode 803 and further filtered by capacitor 805. In a one instance, diode 803 can be implemented using a diode having a low forward bias and fast switching capabilities, such as a Schottky diode.

In a particular embodiment of the present invention, RF transmission 801 contains a power component for charging battery 815 and a signal component for controlling LED 825. Capacitor 805 can be selected to separate these components for use by the circuit. For instance, the power component may be a relatively low-frequency, large-amplitude signal, while the signal component is a relatively high-frequency, small-amplitude signal. Capacitor 805 can be selected to filter the power component of the signal to create a corresponding voltage. The remaining the high-frequency component of the RF transmission is added to this voltage. The power component of the transmission can then be used to charge on the battery 815, and the signal component of the transmission is used to enable LED 825. The light generated by LED 825 to triggers stimulus of the target cells 827.

FIG. 8B illustrates an alternative embodiment radio-frequency energy accumulator, which charges a battery, which in turn, powers a digital pulse generator, which powers a LED. An electromagnetic signal 850 is received by loop antenna 852 generating a corresponding electrical signal. The voltage generated from loop antenna 852 is limited by the reverse bias voltage of the diodes 855 and 856 and stored in capacitor 854. In a particular instance these diodes have a low reverse bias voltage that is relatively precise, such as a Zener diode. Electromagnetic signal 850 is rectified via diode rectifier bridge 858 and filtered by voltage regulator 859 to produce a DC voltage. The DC can be used to charge power source 860.

Battery 860 is coupled to the input of Schmidt trigger 865 through capacitor 862. Feedback from the output of the Schmidt trigger is provided through resistor 864 relative to the charge on capacitor 863. Accordingly, the frequency of the square-wave output of Schmidt trigger 865 is determined by the values of the resistor-capacitor network including capacitor 863 and resistor 864. Resistor 864 and capacitor 863 may be fixed or variable. The output of Schmidt trigger 865 is fed through digital inverter 867 which powers LED 866. Light from LED 866 is transmitted to light-sensitive neurons 868 relative to the frequency of the square-wave output of Schmidt trigger 865.

FIG. 8C illustrates block diagram for an electromagnetic filed (EMF) energy accumulator and pulsing approach in which the received EMF 897 (for example radiofrequency energy) includes not only energy for accumulation, but also an encoded signal regarding instructions to microcontroller 895. In step 885 (Energy plus Parameter Control Signal: Encoding and transmission), a control instruction signal is encoded to ride upon the energy component by methods known in the art, for example, by frequency modulation. Energy receiver block 890 uses a portion of the EMF signal to provide power to block 893. Control signal receiver block 891 uses a portion of the EMF signal to provide control instructions to microcontroller block 895.

The control instruction can be used to transmit information regarding the various parameters of the generated light, such as frequency, strength, duration, color, and the like. These instructions can be decoded and processed using a microcontroller or logic circuitry as shown by block 895. Block 895 can generate control signal(s) in response to the decoded instructions. Accordingly, the frequency (and other parameters) of the light generated by LED 896 rate need not be fixed for the given implanted device. Antenna 889 delivers input to the Energy Receiver 890 (providing power to voltage regulator and battery circuitry 893). Concurrently, antenna 889 delivers encoded data to Control Signal Receiver 891, which provides control input to microcontroller 895 that drives LED 896. Selected wavelength light 897 is then delivered to electrically excitable cell 898. The battery in the voltage regulator and battery circuitry 893 provides power to the microcontroller 895 and the Control Signal Receiver 891.

The circuit diagrams of FIG. 7 and FIGS. 8A, 8B and 8C are merely illustrative of a few particular embodiments of the present invention, and various other implementations are envisioned. For example, particular embodiments implement a light source that uses a blue LED; however, other colors and light sources can be implemented depending upon the particular application.

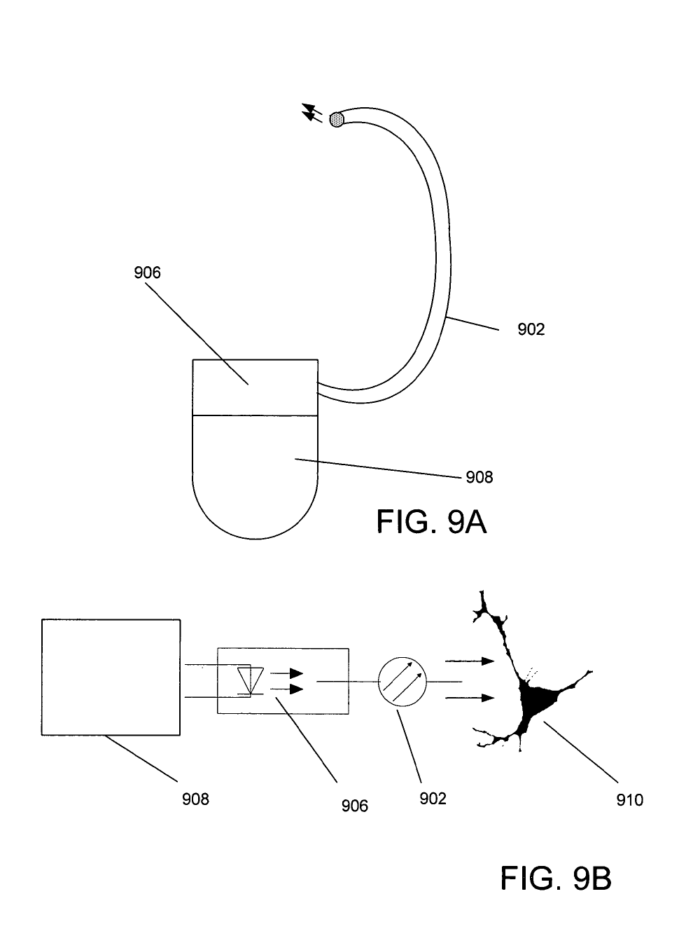

FIG. 9A and FIG. 9B each show a diagram of a fiber-optic device, according to an example embodiment of the present invention. The fiber-optic device includes a control portion 908, a light generator 906 and a fiber optic cable 902.

Fiber optic cable 902 can be positioned near a photosensitive biological portion, such as a viral matrix or synthetic mesh as discussed herein. This allows for control portion 908 and light generator 906 to be located at a distance from the target cells 910 (e.g., at a distance corresponding to the length of fiber-optic cable 902). This can be particularly useful for minimizing the size of the portion of the implanted device that is near the target cells, for example, where the target cells are located at or near a sensitive location within the brain. In some instances, the remote location of portions 908 and 906 also facilitates modifications of the device, including, but not limited to, replacement of various components (e.g., batteries), changes in stimulation frequency and length. Control portion 908 can be configured to respond to an external signal, such as magnetic field or RF signals. Alternatively, control portion 908 can be configured to enable light generator 906 according to a programmed schedule or a combination of an external signal and a programmed response.

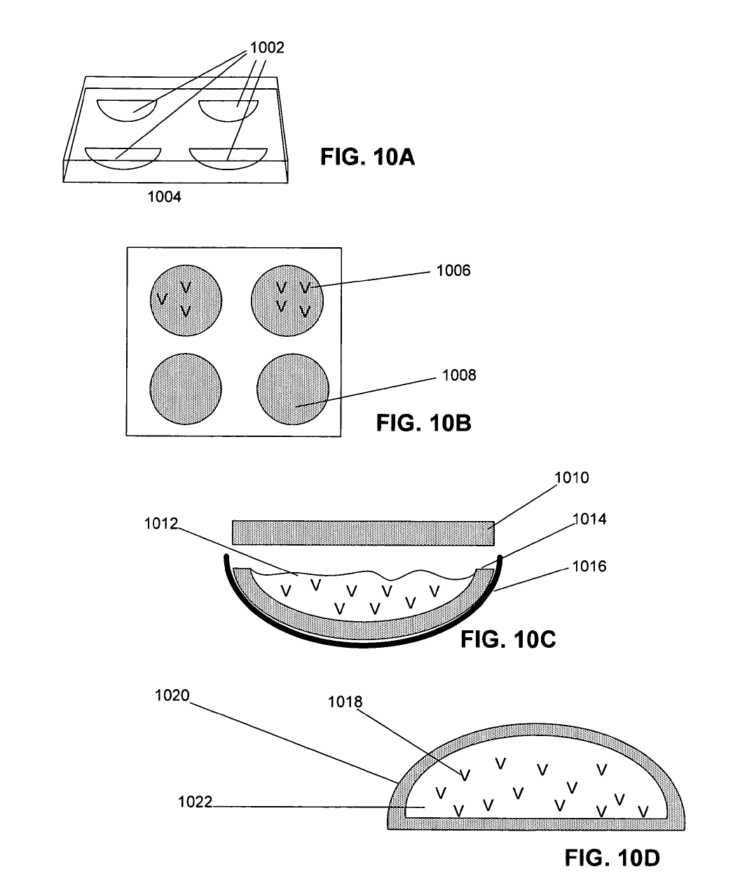

FIGS. 10A-10D depict various stages in the production of a photosensitive biological portion, according to an example embodiment of the present invention. More specifically, FIG. 10A shows molding structure 1004 having several molds 1002. Molds 1002 are constructed to various sizes depending upon the particular application. In one such application, the molds are a few millimeters or less in diameter.

FIG. 10B shows the molds 1002 from FIG. 10A after applying a layer of gelatin or similar substance as shown by 1006 and 1008. Moreover, viral vectors (shown by ‘v’) are in the upper two molds. These viruses may be suspended within media 1012, which may be a liquid or gelatinous media. Such liquids include normal saline, HEPES-buffered saline and other known viral sustenance and transfer media. Suitable gelatinous media includes Matrigel (BD Biosciences, San Jose, Calif.) These viral vectors are designed transfer genes for light-sensitization to the membranes of targeted cells after implantation.

FIG. 10C shows a side view of mold 1006. 1016 represents the molding structure that forms the shape of gelatin layer 1014. Gelatin layer 1014 traps viral vectors contained within media 1012. A top gelatin layer 1010 is applied to fully contain the viral vectors.

FIG. 10D shows the resulting viral vector capsule. The viral vectors 1018 are contained within area 1022 by casing 1020. Casing 1020 can be designed to dissolve or otherwise allow viral vectors 1018 to disseminate towards the target cells once implanted. In one instance, the capsule is constructed of a water soluble material, for example, gelatin, so that upon implantation the viral vectors are allowed to escape into the body. Water soluble capsule materials are well known in the pharmaceutical industry.

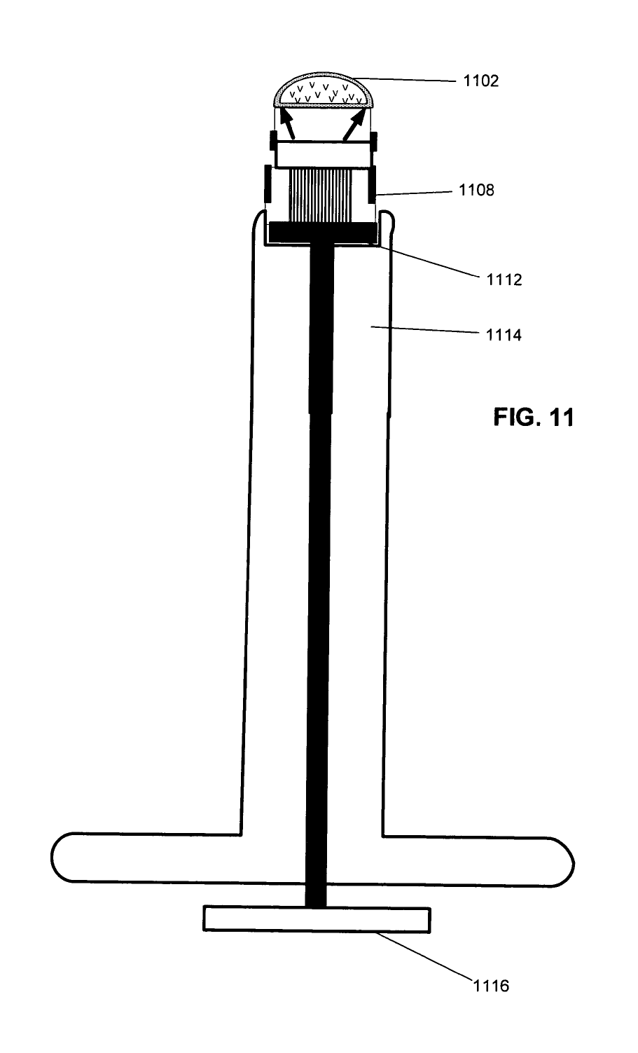

FIG. 11 shows an implantation device, according to an example embodiment of the present invention. Biological portion 1102 and light generation device 1108 are implanted using the implantation device. For example, the shaft of the device 1114 is positioned near the target cells. Next, a user of the device presses on portion 1116 which causes portion 1112 to place biological portion 1102 and light generation device 1108 near the target cells. The implantation device can then be removed.

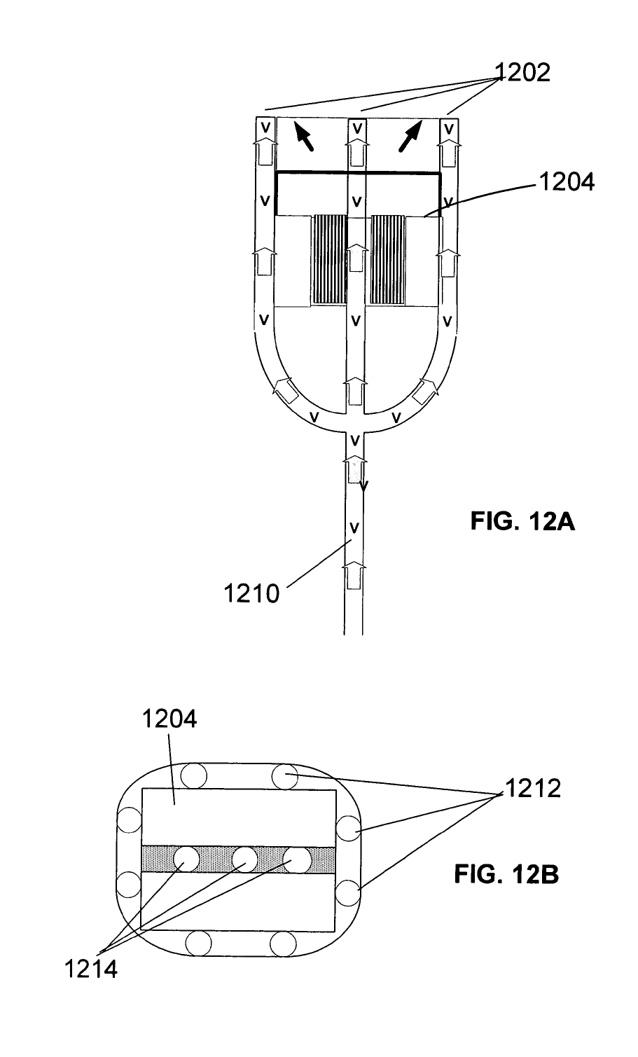

FIG. 12A and FIG. 12B show a diagram for another implantation device, according to an example embodiment of the present invention. Implantable light generating device 1204 is surrounded by, and permeated by fluid channels 1202. Fluid channels 1202 allow a solution 1210 containing bio-molecular material (e.g., photosensitizing viral vectors) to be injected immediately proximal to light generating device 1204 and the target cells. The fluid channels can be located outside of device 1204 and/or within device 1204, as shown by 1212 and 1214 respectively. In this manner, the viral vectors can be injected in large quantities or over a period of time. For instance, cells infected by viral vectors can revert back to their pre-infection state after a period of time. Using the device of FIG. 12A, the viral vectors can be periodically reintroduced to the target cells. Alternatively, different viral vectors can be introduced through the fluid channels, allowing for targeting of different cells at the implantation site. This can be particularly useful for staged treatment through stimulation of different types of cells.

A specific embodiment of the present invention relates to a method for genetically modifying neurons to express light-sensitive ion channel ChannelRhodopsin (ChR2). In this method, pulses of blue light causes ChR2 neurons to fire action potentials corresponding to each pulse. Depolarization and repolarization occur on a millisecond timescale making this method consistent with normal network neurophysiology.

Specific targeted neurons are modified using viral vectors for gene transfer. For further details on the generation of viral vectors reference can be made to Boyden et al 2005, Zhang et al 2006, both of which are fully incorporated herein by reference. This transfection results in the introduction of a gene for a single protein, a cell membrane ion channel, known as “Channelrhodopsin 2”, or “ChR2”. In nature, ChR2 resides on the cellular membrane of unicellular green algae Chlamydormas reinhardtii. Upon absorption of blue light (470-480 nm), this ion channel briefly opens, allowing cation influx. When transfected into a mammalian nerve cell, affected nerves become photosensitive, producing light-triggered action potentials. To produce this action potential, photosensitized nerves appear to require 5-10 mW/mm of blue light intensity, in flashes up to 30 Hz. In experimental conditions, 98% of the time, such a flash of light produces an action potential within 50 μseconds of the flash, with a variability (jitter) of 5 μseconds.

A neuronal-type specific feature which is also a robust promoter (for example, CaMKIIa) is inserted adjacent to the ChR2 code within the virus, and the line is propagated by calcium-phosphate cotransfection of 293FT cells. The supernatant is then centrifuged into viral pellets, which are placed within phosphate-buffered saline.

In a particular instance, application of an algal light-gated ion channel Channelrhodopsin-2 is used for photostimulation. The first 315 amino-acid residues of the algal Channelrhodopsin-2 (abbreviated as ChR2 when coupled with retinal, or Chop-2 for the gene) from Chlamydomonas reinhardtii can be used to impart fast photosensitivity upon mammalian nerve cells, by using a viral vector to insert the gene for ChR2 into targeted nerve cells which may subsequently express this gene. ChR2 is a seven-transmembrane protein with a molecule of all-trans retinal (ATR) bound at the core as a photosensor. Upon illumination with approximately 470 nm blue light, ATR isomerizes and triggers a conformational change to open the channel pore. As ChR2 is a light-sensitive ion channel, it allows an inward current to be evoked within 50 μs of illumination. Combining ChR2 with ultrafast light switching it is possible to activate neurons at the temporal precision of single action potentials, reliably over sustained multiple action potential trains.

In another instance, application of bacterial light-gated chloride channel halorhodopsin (NpHR) is used for photostimulation. This ion channel can be imparted upon mammalian nerve cells by using a viral vector to insert the gene for NpHR into targeted nerve cells, which may subsequently express this gene. Upon illumination with approximately 550 to 626 nm amber light, active pumping of chloride ions into the neuronal cytoplasm results in hyperpolarization of the cell.

For each application, the underlying physical properties of the native signal can be considered when choosing the most suitable of these described photostimulation methods. Excitable cells distinguish inputs in part based on their temporal properties, channel recruitment patterns and amplitude or polarity characteristics. Regarding temporal properties, glutamate uncaging and ChR2 achieve responses on the millisecond time scale. Such responses are well suited for photostimulating pathways triggered by fast synaptic events and action potentials. Regarding channel recruitment patterns, glutamate uncaging directly activates native glutamate receptors and so may achieve physiological spatial patterns of subcellular excitation. However, the other photostimulation methods, via depolarization, will recruit native voltage-activated channels such as voltage-dependent calcium, sodium and potassium channels, and thereby activate native, spatially sensitive signaling pathways. With such methods, channels could be activated experimentally so that populations can be labeled via stereotactic injection of viruses that effect retrograde axonal transport, by taking advantage of region specific axonal projections. Just as with ChR2, other genetically based photostimulation methods (including NpHR) can use these targeting strategies, although some multicomponent systems may be difficult to implement without the use of transgenic technologies. For a photostimulation-based method, sufficient gene expression must be achieved to elicit physiologically relevant levels of current.

In a particular instance, ChR2 is activated with blue light (excitation around 470 nm). Successful photostimulation of ChR2-expressing cells requires at least 5 mW/mm2 of blue light to reach the sample.

ChR2 has been estimated to possess a single-channel conductance as low as 50 femtosiemens. This would imply that between 100,000 and 1,000,000 ChR2 molecules would have to be generated and localized to the neuronal membrane to achieve the observed currents in the range of 1 nA (starting from a resting potential of −70 mV and neglecting space-clamp issues and changes in driving force due to ion entry).

Since sensitivity to blue light via ChR2 is induced when a viral vector inserts the ChR2 gene into a previously normal cell, the insertion may be genetically targeted to the products expressed by specific cellular subtypes. For example, it might be advantageous to cause only dopaminergic neurons, and not cholinergic neurons to react to blue light. The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. For instance, such changes may include the use of digital logic or microprocessors to control the emitted light. Such modifications and changes do not depart from the true spirit and scope of the present invention, which is set forth in the following claims.

1. An implantable arrangement for in vivo use, comprising:
a light generator for generating light; and
a biological portion that modifies target cells to facilitate stimulation of the target cells in response to light generated by the light generator.

2. The implantable arrangement of claim 1, wherein the implantable arrangement is scopically-deliverable.

3. The implantable arrangement of claim 1, wherein the implantable arrangement is stereotactically deliverable.

4. The implantable arrangement of claim 1, wherein the biological portion modifies the target cells to produce a light-activatable ion channel protein in the target cells.

5. The implantable arrangement of claim 4, wherein a light-activated ion channel is coded for by a virus configured to infect the target cells.

6. The implantable arrangement of claim 4, wherein the light activated ion channel protein one of Channelrhodopsin-2, Chop2, ChR2-310 and Chop2-310.

7. The implantable arrangement of claim 1, wherein the light generator comprises a light-emitting-diode responsive to an input signal.

8. The implantable arrangement of claim 1, wherein the biological portion comprises a viral matrix containing viral vectors.

9. The implantable arrangement of claim 1, wherein the biological portion comprises a membrane layer containing viral vectors.

10. The implantable arrangement of claim 5, wherein the light-activated ion channel is selected from the group consisting of Channelrhodopsin-2, Chop2, ChR2-310 and Chop2-310.

11. The implantable arrangement of claim 5, wherein the light-activated ion channel is halorhodopsin.

12. The implantable arrangement of claim 1, wherein the target cells are neurons.

13. The implantable arrangement of claim 1, wherein the stimulus includes electrical current due to depolarization of the target cell.

14. An implantable device for stimulating target cells modified to have a photosensitive bio-molecular structure, comprising:
a light generator to generate light in response to an external signal; and
a biological arrangement that modifies the target cells to include the photosensitive bio-molecular structure.

15. The device of claim 14, wherein the photosensitive bio-molecular structure is a light-activatable ion channel protein.

16. The device of claim 14, wherein the photosensitive bio-molecular structure is Channelrhodopsin-2.

17. The device of claim 14, wherein the photosensitive bio-molecular structure is a light-activatable charged-ion membrane channel.

18. The device of claim 14, wherein the photosensitive bio-molecular structure is Chop2.

19. The device of claim 14, wherein the photosensitive bio-molecular structure is ChR2-310.

20. The device of claim 14, wherein the photosensitive bio-molecular structure is Chop2-310.

21. The device of claim 14, wherein the photosensitive bio-molecular structure is halorhodopsin.

22. The device of claim 14, further comprising a signal-reception circuit, electrically coupled to the light generator, to respond to the external signal.

23. The device of claim 22, wherein the external signal is an electromagnetic transmission, and wherein the signal-reception circuit comprises at least one coil and a light-emitting diode, said at least one coil and the light-emitting diode being activated to provide the generated light in response to the electromagnetic transmission.

24. The device of claim 22, wherein the external signal is a radio-frequency (RF) transmission, and wherein the signal-reception circuit includes an RF receiver being activated to provide the generated light in response to the radio frequency transmission.

25. The device of claim 23, wherein the electromagnetic transmission includes a power portion of the transmission that charges a power source and a signal portion of the transmission that is used to enable or disable the light generator.

26. A method for stimulating target cells using a photosensitive protein that allows the target cells to be stimulated in response to light, the method including:
implanting vectors that modify the target cells to include the photosensitive protein;
implanting a light generating device near the target cells; and
activating the light generating device to generate light to be received by the target cells, thereby stimulating the target cells in response to the generated light.

27.-37. (canceled)

38. The implantable arrangement of claim 1, wherein the biological portion comprises a containment material that comprises a nucleic acid comprising a nucleotide sequence encoding a light-responsive opsin polypeptide.

39. The implantable arrangement of claim 38, wherein the light-responsive opsin polypeptide is a channelrhodopsin comprising an amino acid having at least 95% amino acid sequence identity to SEQ ID NO:1.

40. The device of claim 14, wherein the photo-sensitive bio-molecular structure comprises a light-responsive opsin polypeptide.

41. The device of claim 40, wherein the light-responsive opsin polypeptide has at least 95% amino acid sequence identity to SEQ ID NO:1.
